(19) United States
(12) Patent Application Publication
(10) Pub. No.: US 2023/0271232 A1
(43) Pub. Date: Aug. 31, 2023

(54) SYSTEM AND METHOD FOR PROTECTING PROTECT VALUABLE, SENSITIVE, OR ELECTRO-MECHANICAL EQUIPMENT DURING CLEANING OR DISINFECTING

(71) Applicant: SONABLATE CORP., CHARLOTTE, NC (US)

(72) Inventors: Ralf Seip, Pawling, NY (US); Grant Adam Morris, Frankfort, IN (US); Kelly Ledbetter, Carmel, IN (US); Ashok Mehta, Indianapolis, IN (US); Joshua Huff, Indianapolis, IN (US)

(21) Appl. No.: 17/652,604

(22) Filed: Feb. 25, 2022

(51) Int. Cl. B08B 11/00; A61L 2/18

(52) U.S. Cl. CPC B08B 11/00; A61L 2/18; B08B 2203/0264; A61L 2202/24; A61L 2202/121

(57) ABSTRACT

A system configured to protect valuable, sensitive, or electro-mechanical equipment during cleaning of the equipment can include a cover having a single opening leading to an interior cavity. The cover can be waterproof. A plate can be permanently attached to the cable of the equipment. An outer periphery of the plate can be approximately the same size as an inner periphery of the opening. The plate can be configured to sealingly fit within the single opening. The system can include a latching mechanism configured to sealingly attach the plate to the cover.

### FIELD

The presently disclosed technology relates generally to processing electro-mechanical devices and equipment, such as a portion of a medical device. More specifically, in one embodiment, the presently disclosed technology relates to simplifying the washing or cleaning of valuable, sensitive, and/or electro-mechanical devices.

### BACKGROUND AND DESCRIPTION OF RELATED ART

It can be important to keep complex, sensitive, or expensive equipment clean for optimum performance and to extend the working lifespan thereof. For example, medical devices are often cleaned or high-level disinfected (HLD) prior to or after use by an automated machine-based washing cycle. The benefits of an automated machine-based washing cycle include increased repeatability of the process and reduced time as compared to manual cleaning.

Machine washing (or more generally, “reprocessing”) of electro-mechanical devices poses a special challenge. The mechanical components of an electro-mechanical device may be compatible with the machine wash process. However, the electrical parts of an electro-mechanical device (e.g., connectors, ports, sensors, etc.) may not be compatible with the machine wash process.

One prior art connector enclosure designed to protect the connector of a medical device is the SCOPEVAULT™ by NEOCARE™, which is based in the United Kingdom. The SCOPEVAULT™ design employs a hard-shelled enclosure and a connector cable sealing mechanism that seals around the connector cable, allowing the connector itself to be fully enclosed by the hard enclosure. The SCOPEVAULT™ design relies on the user pressurizing the enclosure prior to each wash cycle. It is this pressure interacting with the cable sealing mechanism that seals the entire enclosure around the connector and connector cable. This is analogous to a car's tire pressure, where a force is required to push against the inflation valve from the inside to keep the valve closed and the tire inflated.

Without the increased pressure described above, the cable sealing mechanism of the SCOPEVAULT™ design will not reliably seal around the connector cable, which will result in water or cleaning/disinfecting fluids entering into the connector enclosure during the wash/reprocessing cycle, negating the protection that the connector cover is supposed to provide. The presence of this elevated pressure in the SCOPEVAULT™ design will also deter fluid entry into the connector cover during the wash cycle, as any water/fluids that would potentially breach the seal and make its way into the enclosure would have to overcome this internal pressure, which is an unlikely occurrence. While reliable, the connector enclosure of the SCOPEVAULT™ design relies on additional steps to be performed by the user, which if not accomplished and checked correctly, may lead to fluid entry and connector exposure and possible connector damage during the wash cycle.

The connector enclosure of the SCOPEVAULT™ design also requires the connector-connector cable-medical device assembly (or its individual components) to be fully leak-proof and resistant to the same pressures that the connector cover would be experiencing. Otherwise, pressure in the connector cover will leak through the connector and connector cable and reach the device itself, potentially pressurizing the inside of the device to the same pressure as that found in the connector cover required to seal it, which may lead to device damage.

### BRIEF SUMMARY

Despite the benefits of the prior art, there is a need for an improved system that more easily and efficiently protects one or more components of medical devices (e.g., the electrical components thereof) and other sensitive equipment during the machine wash process and any other cleaning.

The presently disclosed technology overcomes the above and other drawbacks of the prior art in both the medical device and other arts.

In one optional embodiment, the presently disclosed technology is directed to protecting connectors and/or ports, for example, of electro-mechanical equipment by use of a connector enclosure. The enclosure is configured to protect the connectors and/or ports from 1) the fluids (e.g., water, enzymatic cleaners, disinfection solutions, etc.) used during the wash cycle, 2) the temperature cycles to which the connector is exposed to during the wash cycle (typically ranging from room-temperature to 80+° C. and back down to room-temperature), and 3) the high-pressure fluid spray and spray nozzles the connector would otherwise be exposed to during the wash/reprocessing cycle.

In another optional embodiment, the presently disclosed technology is directed to a system configured to protect valuable, sensitive, or electro-mechanical equipment during cleaning of the equipment can include a cover having a single opening leading to an interior cavity. The cover can be waterproof. A plate can be permanently attached to the cable of the equipment. An outer periphery of the plate can be approximately the same size as an inner periphery of the opening. The plate can be configured to sealingly fit within the single opening. The system can include a latching mechanism configured to sealingly attach the plate to the cover.

In yet another optional embodiment, the presently disclosed technology is directed to a combination including a medical device having a cable and a system configured to protect the medical device during cleaning. The system can include a cover having a single opening leading to an interior cavity. The cover can be waterproof. A plate can be permanently attached to the cable of the equipment. An outer periphery of the plate can be approximately the same size as an inner periphery of the opening. A latching mechanism can be configured to sealingly attach the plate to the cover. At least one of a connector or a port of the medical device is configured to be positioned in the interior cavity of the cover. The plate can be configured to be inserted into and sealingly engage the single opening of the cover and enclose at least one of the connector or the port of the medical device within the interior cavity. The cable can extend through the plate in a watertight manner.

### BRIEF DESCRIPTION OF THE DRAWINGS

The foregoing summary, as well as the following detailed description of the presently disclosed technology, will be better understood when read in conjunction with the appended drawings, wherein like numerals designate like elements throughout. For the purpose of illustrating the presently disclosed technology, there are shown in the drawings various illustrative embodiments. It should be understood, however, that the presently disclosed technology is not limited to the precise arrangements and instrumentalities shown. In the drawings:

FIG. 1 is a perspective view of a system according to an embodiment of the presently disclosed technology, wherein a portion of valuable, sensitive, or electro-mechanical equipment is shown;

FIG. 2 is a perspective view of a cover or enclosure of the system shown in FIG. 1;

FIG. 3 is a perspective view of a second portion of the system shown in FIG. 1, wherein a portion of the valuable, sensitive, or electro-mechanical equipment is attached to a cover of the system;

FIG. 4 is a perspective view of a system according to another embodiment of the presently disclosed technology;

FIG. 5 is another perspective view of a portion of the system shown in FIG. 4;

FIG. 6 is a schematic cross-sectional side elevation view of a portion of the system shown in FIG. 4;

FIG. 7 is a system according to yet another embodiment of the presently disclosed technology; and

FIG. 8 is another perspective view of the system shown in FIG. 7.

### DETAILED DESCRIPTION

While systems, devices and methods are described herein by way of examples and embodiments, those skilled in the art recognize that the presently disclosed technology is not limited to the embodiments or drawings described. Rather, the presently disclosed technology covers all modifications, equivalents and alternatives falling within the spirit and scope of the appended claims. Features of any one embodiment disclosed herein can be omitted or incorporated into another embodiment.

Any headings used herein are for organizational purposes only and are not meant to limit the scope of the description or the claims. As used herein, the word “may” is used in a permissive sense (i.e., meaning having the potential to) rather than the mandatory sense (i.e., meaning must). Unless specifically set forth herein, the terms “a,” “an” and “the” are not limited to one element but instead should be read as meaning “at least one.” The terminology includes the words noted above, derivatives thereof and words of similar import.

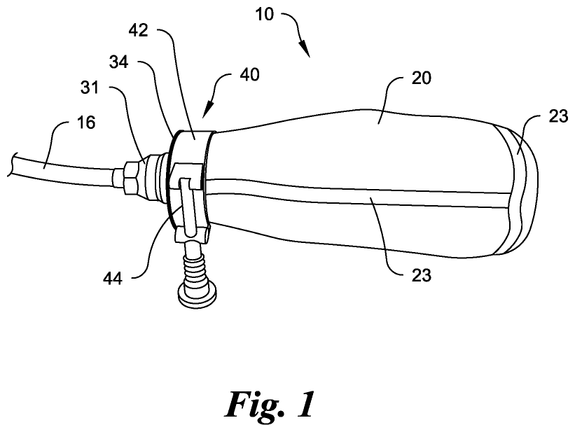

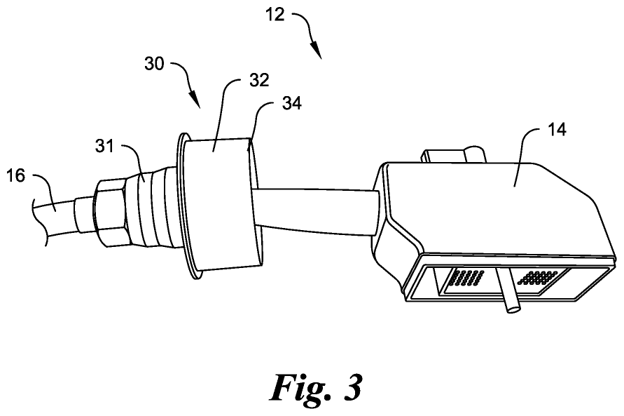

Referring now in detail to the figures, wherein like reference numerals refer to like parts throughout, FIGS. 1-3 show a system, generally designated 10, configured to protect valuable, sensitive, or electro-mechanical equipment, a portion of which is generally designated 12 in FIG. 3, during processing and/or cleaning. The processing and/or cleaning can include but is not limited to i) washing, such as by hand and/or an automated machine, ii) disinfection, such as through a high-level disinfection cycle, and/or ii) device reprocessing. Device reprocessing can involve submerging the entire device in a cleaning/disinfection solution and then leaving the device in the solution for a predetermined amount of time to allow the solution to achieve its desired effect.

In one embodiment, the system 10 is configured to keep at least a portion of the equipment 12 protected and/or dry during a cleaning and/or high-level disinfection cycle completed with a clinical machine washer. Optionally, this can be accomplished without pressurizing the system, which is contrary to what is done in the prior art to manage fluid ingress, thereby simplifying the equipment preparation process prior to machine wash.

As shown in FIGS. 1 and 3, the equipment 12 can include at least one connector or port 14 (see FIG. 3), and a cable 16 electrically connected thereto and extending therefrom. In one embodiment, the equipment 12 can be a medical device, such as a high intensity focused ultrasound (HIFU) probe or other types of probes, an endoscope, a bronchoscope, a colonoscope, or a catheter. The equipment 12 can optionally be any electro-mechanical device that benefits or requires covering or protecting a connector. The configuration of the system 10 allows it to be compatible with equipment 12 of different sizes and shapes, and/or cables 16 having varying diameter sizes.

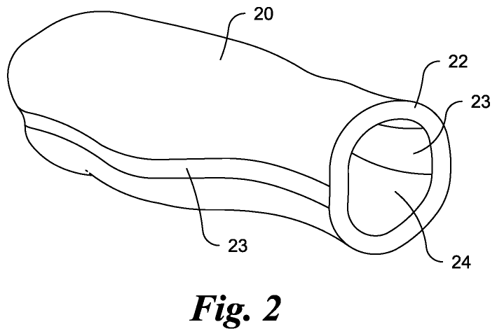

Referring to FIGS. 1 and 2, the system 10 can include an enclosure or cover 20. The cover 20 can be flexible, deformable, and/or compliant, and in one embodiment can be sock-like or bulb-like. The cover 20 can have a single opening 22 (see FIG. 2) leading to an interior cavity 24 therein. In one embodiment, the opening 22 is circular. The cover 20 can be sufficiently larger to hold a connector 14, for example, that needs to be protected during the machine washing cycle.

More particularly, in one embodiment, the cover 20 is waterproof (or fluid proof) and/or formed of a material that is compatible with the chemicals and/or temperatures used during the machine washing cycle. For example, the cover 20 can be formed of one or more of silicone, a fluoroelastomer, nitrile, neoprene, ethylene propylene diene monomer (EPDM) rubber, and a thermoplastic elastomer (TPE). The unique make-up and configuration of the cover 20 helps manage air pressure changes from the reprocessing process inside the equipment 12 that may result in fluid ingress during the wash cycle.

As shown in FIGS. 1 and 2, the cover 20 can include one or more connection or sew lines 23. Optionally, one of the lines 23 can extend around the periphery of the cover 20, and another of the lines 23 can extend parallel to a longitudinal axis of the cover 20. The lines 23 optionally form a permanent (e.g., waterproof or fluid proof) seal. In one embodiment, the material and fabrication used to form the cover 20 and/or the connection line(s) 23 is the same as or similar to that used to make a waterproof wetsuit.

A plate 30 can be attached to the cable 16 of the equipment 12. The cable 16 can extend through the plate 30, such as through a geometric center thereof, in a watertight manner, optionally via a seal or gland structure 31 or an equivalent thereof, such as the HSK-INOX-HD from Hummel AG of Germany. More particularly, the plate 30 can include a central opening configured to sealing receive at least a portion of the 16 cable therein. In one embodiment, the plate 30 is permanently attached to the cable 16. The junction of the plate 30 and the cable 16 tends to typically be susceptible to fluid ingress and requires special considerations for sealing. In one embodiment, the plate 30 can be formed of a polymeric material.

As shown in FIG. 3, an outer periphery of the plate 30 can be circular. The outer periphery of the plate 30 can be approximately the same size as an inner periphery of the single opening 22 of the cover 20, which means that the plate 30 can be designed to easily mate with the single opening 22 of the cover. Optionally, the outer periphery of the plate 30 can be slightly smaller than the inner periphery of the single opening 22, so that the plate 30 is unobtrusive and the single opening 22 can be squeezed or contracted to firmly grip the outer periphery of the plate 30. Thus, in the context of the size of the plate 30, the term “approximately” is defined herein to be any size or difference in size that allows the plate 30 to be securely attached to the cover 20 in a watertight or fluid tight manner.

Referring again to FIG. 3, the outer periphery of the plate 30 can include a groove 32. The groove 32 can extend around the entire perimeter of the plate 30. Optionally, the groove 32 can be formed by or between a slightly raised wall or projection 34 at one or both a front face and a rear face of the plate 30. As explained in further detail below, the groove 32 can be configured to facilitate securing the plate 30 to the cover 20.

At least one latching mechanism 40 can be configured to sealingly attach the plate 30 to the cover 20. The latching mechanism 40 can be easy or simple to operate, and enables a fluid-tight coupling of the cover 20 and the plate 30. Optionally, the latching mechanism 40 can include a collar 42 that is sized, shaped, and or configured to extend around the plate 30 and complement or matingly engage the groove 32. A fastener 44 can be configured to clamp or tighten the collar 42 in the groove 32 and around the plate 30. A longitudinal axis of the fastener 44 can extend perpendicularly to a longitudinal axis of the cable 16 when the connector or port 14 is positioned inside the cover 30.

In operation, as shown in FIG. 1, at least one of the connector or port 14 of the equipment 12 can be configured to be positioned in the interior cavity 24 of the cover 20. The single opening 22 may need to be elastically expanded to allow the connector or port 14 to pass therethrough, before returning to its original size or configuration. The plate 30 can be configured to be inserted into and sealingly engage the single opening 22 of the cover 20 and enclose at least one of the connector or the port 14 of the equipment 12 within the interior cavity 24. The latching mechanism 40 can be attached to ensure the fluid-tight connection between the cover 20 and the plate 30.

In one embodiment, the presently disclosed technology relies on the plate 30 to be permanently attached to the cable 16. A significant number of studies have shown that this is the best and most reliable solution if the cover 20 is going to be used exclusively for a single cable diameter/design. Unlike the currently existing general-purpose enclosures, which require pressurization to operate correctly (and are therefore expensive), the simple and cost-effective design of the presently disclosed technology allows for each equipment 12 to have its own, dedicated, permanent cover 20, which could remain with the equipment 12 at all times.

The plate 30 can be manufactured via standard means (e.g., machining, sheet metal bending, welding, polishing, etc.). The plate 30 and the seal or gland 31 can then be mated with the cable 16 of the connector 14. This can occur during manufacturing of the cable 16 or can occur after the manufacturing of the cable 16 has been completed, to retrofit cables that have already been released and are being used in the field. From this point forward, the plate 30 remains firmly attached to the cable 16.

Alternatively, the cover 20 can also be installed around the connector 14 to protect it during a manual cleaning/washing/disinfection step. In this case, it simply prevents the connector 14 from accidentally being exposed to any liquids during the manual cleaning step.

To protect the connector 14 from the clinical machine washer, all the user now needs to do is to place the connector 14 in the cover 20 (through the opening 22), correctly align the plate 30 with the cover 20, and activate or engage the latching mechanism 40. The system 10 is now ready for a machine wash cycle, and the cover 20 will maintain the connector 14 protected and dry. After the washing cycle, the cover 20 can be removed, or it can simply stay in place, until it needs to be removed prior to use of the equipment 12 and/or connector 14.

There are numerous benefits of the present design. For example, the system 10 is compact or small. The system 10 does not rely on pressurizing the cover 20 or the other components of the system 10 or the equipment 12 to be pressure-proof, because the equipment 12 can optionally be cleaned with the system 10 without pressurizing the interior cavity 24 of the cover 20. The entire system 10 and equipment 12 can remain at the same pressure throughout the cleaning cycle. The system 10 does not require the cable 16 to prevent air from moving from the cover 20 to the equipment 12. The cover 20 is capable of expanding to accommodate any pressure caused by temperature changes during washing and/or drying.

The compliant nature of the cover 20 prevents air pressure build-up during the machine wash cycle (example: a temperature rise from 25° C. to 70° C.—typical during a machine wash cycle, will increase the pressure inside the enclosure/connector/cable device assembly by approximately 2.2 psi if the volume of this structure remains the same). The pressure increase would manifest itself instead as a volume increase, which the compliant closure 20 is able to accommodate. The cover 20 can be implemented in the form of a single-use or disposable part, which would make the cover 20 simple and cost-effective to manufacture.

The compliant nature of the cover 20 allows for keeping the plate 30 size manageable (e.g., relatively small), as it allows the single opening 22 to expand and stretch over the connector 14 during attachment, and then return to its upstretched size when mating with the plate 30. This allows for the plate 30 to be of the same or approximately the same cross-section size of the connector 14 or even a bit smaller, which is always advantageous from a cost, manufacturing, and weight perspective.

Further, the circular geometry of the plate 30 and the single opening 22 of the cover 20 allow the user to mate the cover 20 with the plate 30 at any angle, thereby simplifying the preparation steps for machine washing/device reprocessing.

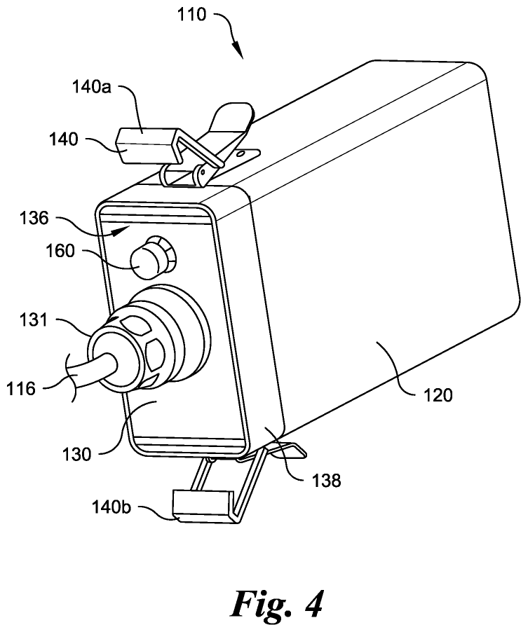

FIGS. 4-6 show another embodiment of the system, generally designated 110, of the presently disclosed technology. Similar or identical structure as between the embodiment of FIGS. 1-3 and the embodiment of FIGS. 4-6 is distinguished in FIGS. 4-6 by a reference number with a magnitude one hundred (100) greater than that of FIGS. 1-3. Description of certain similarities between the embodiment of FIGS. 1-3 and the embodiment of FIGS. 4-6 may be omitted herein for convenience and brevity only.

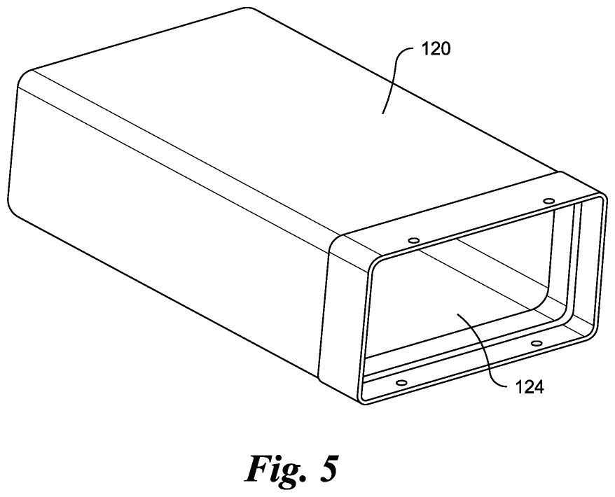

One distinguishing feature of the embodiment of FIGS. 4-8 is that the cover 120 is rigid, hard, and/or formed of a metallic material. Unlike the sock-like or bulb-like configuration of the prior embodiment, the cover 120 can have a square, rectangular, or rectilinear outer shape, and a similarly shaped single opening 120 and/or interior cavity 124 (see FIG. 5). The cover 120 can have a cross-sectional shape that is the same or matches a cross-sectional shape of the connector or port the cover 120 is designed to protect. The cover 120 can be made from a material that is compatible with the chemicals and temperatures used during the wash cycle (e.g., steel, stainless steel, surgical steel, titanium, or the like). In one embodiment, surgical steel is preferred due to its compatibility with clinical machine washing/device reprocessing cycles, longevity, and hardness (e.g., surgical steel won't bulge when inside pressure is increased).

Similar to the prior embodiment, the plate 130 can receive the cable 116 therethrough, and the two can be attached in a fixed and/or fluid-proof manner. The seal or gland 131, or the equivalent thereto, (see, e.g., FIG. 5) can create the fluid-proof connection. Optionally, the plate 130 can be made out of a plastic-type material (i.e., ULTEM™ or polyethermide, glass-filled ULTEM™, or a similar material) to minimize scratches to adjacent pieces of equipment when handing the system 110. Alternatively, the plate 130 can be formed of steel, stainless steel, surgical steel, titanium, PEI, PPSU, PEEK, and LCP. In one embodiment, ULTEM™ is preferred due to its light weight, machinability, stiffness, availability in many colors (e.g., ULTEM™ can match the cable and connector cover color), and won't scratch other pieces of equipment that the connector would be attached to.

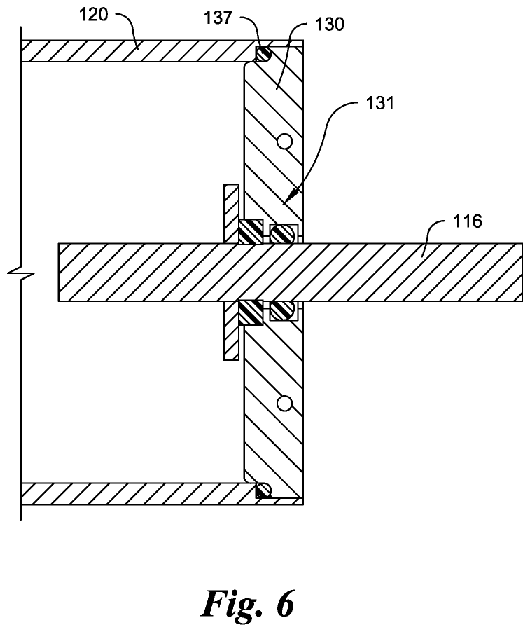

The plate 130, which can have the same cross-sectional shape as the cover 120 and the connector or port, can mate or attach to the cover 120 in a fluid-proof and/or a gas-proof manner through the latch mechanism 140 to generate the compression force needed to create a fluid- and air-tight seal between the plate 130 and the cover 120. As shown in FIG. 6, an outer periphery of the plate 130 can include a circumferential seal 137, such as an O-ring or gasket, to help facilitate the fluid-proof and/or gas-proof connection with the cover 120.

In one embodiment, cover 120 can be fabricated out of two pieces, which can include a precision bezel 138 that will receive the plate 130, and the body of the cover 120, which is welded to the bezel 138. This configuration allows for sheet metal fabrication techniques to be used for the body of the cover 120, while more precise machining fabrication techniques would be used to machine the bezel 138.

The latching mechanism 140 can include a first latch 140a spaced-apart from a second latch 140b. In one embodiment, each latch 140a, 140b is an over-the-center latch that is configured to extend from (and/or be fixedly attached to) the cover 120 and engage or secure to a portion of the plate 130. The plate 130 can include one or more spaced-apart recesses 136 (see FIG. 4) on the front face thereof, which can be sized, shaped, and/or configured to receive at least a portion of one of the first and second latches 140a, 140b therein.

Sealing of the plate 130 in or to the cover 120 can be provided by a circumferential/rectangular seal (e.g., circular cross-section), optionally made out of silicone shore A20 or similar material, which is capable of withstanding the many heating/cooling/wash cycles the cover 120 would be exposed to. Other materials that the seal can be formed of include silicone of various hardnesses for sealability, durability, and compatibility with clinical machine-washing systems (Shore A10-40 typical). Compression of this seal can be achieved by pressing down on the plate 130 with the latching or pin mechanism described above. One alternate materials for seal construction includes PORON™ (polyurethane).

The cover 120 enables an international protection IPX7 (waterproof) rating when coupled to the Sonablate HIFU probe (Sonablate Corp.), protecting the entire probe from fluid ingress during any type of reprocessing, manual or machine-based.

As shown in FIG. 4, the plate 130 can optionally include a valve 160. In an alternative embodiment, the valve 160 can be located in or extend through the cover. Optionally, the valve 160 can be a pressurization valve or a luer valve. Alternatively or additionally, the valve 160 can be configured to test the integrity of the cover 120, the cable 116, and the plate 130.

The luer valve 160 is optional, as the system 110, including the equipment and/or cable 116 therein, can remain at the same (e.g., ambient) pressure throughout the cleaning cycle. In the event that the user wishes the pressurize the internal cavity 124, the luer valve 160 can be used to assist with that process and/or the cover 120 can be pressurized through the luer valve 160 to check the integrity of the system 110. Alternatively, this step is only followed during equipment 112 maintenance by a technician to check the integrity of the system 110, to facilitate the duties of the user who would not typically perform this step. The luer valve 160 can prove useful for verifying (during routine medical device service, for example), if the system 110 is still fluid/air proof, as needed for machine washing compatibility or to ensure fluids won't penetrate the device during normal operation.

The luer valve 160 can be an important aspect of one embodiment of the presently disclosed technology. The luer valve 160 can allow one to not only test the ability of the cover 120 to create the required seal around the connector to protect it during the washing cycle, but also can allow the assembler/technician to test the integrity of the system 110 for leaks. Using the cover 120 this way would enable one to identify cracks in the equipment or breaks in the cable 116 that may lead to water ingress prior to the system 110 being placed in the washer, or as part of a quality inspection procedure during equipment 112 manufacturing. This could be accomplished as follows: place the cover 120 on the connector and seal the system 110. Through the luer valve 160, pressurize the system 110 by using an appropriate tool (e.g., compressed air, syringe, or equivalent). Pressures around 5 psi have shown to be sufficient for this step in one embodiment (e.g., for the case of the Sonablate HIFU probe, from Sonablate Corp.). Next, determine if the system 10 can maintain this pressure over time, monitoring it, for example, with a pressure gauge. If the pressure decreases during a certain time (for example, 5 minutes), this would indicate that there is a leak somewhere in the connector or cable 116, which would compromise the equipment during a wash cycle. Next, after re-establishing the 5 psi pressure inside the system 110, one would place the system 110 inside a waterbath, to determine the location of the leak (which would be indicated by bubbles emanating from it). This process allows the possibility to either repair or discard the equipment and/or the cable 116, as needed, preventing damage in the field. Of course, stable pressure over time indicates no leaks in the cover 120 and integrity for machine washing.

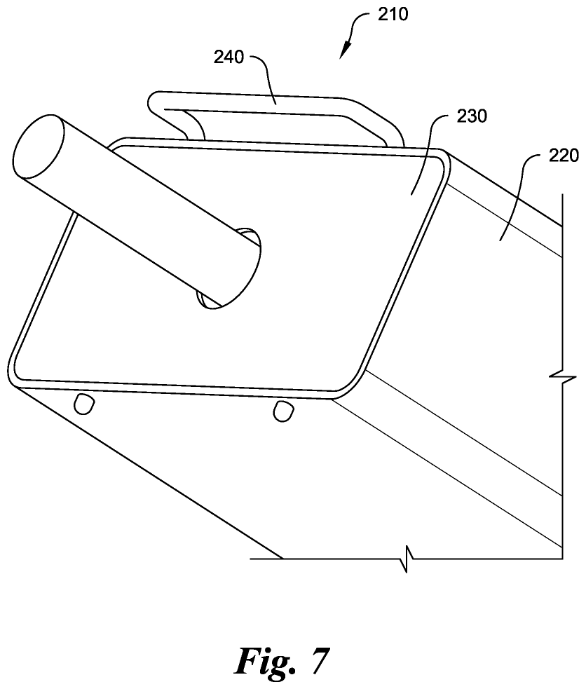

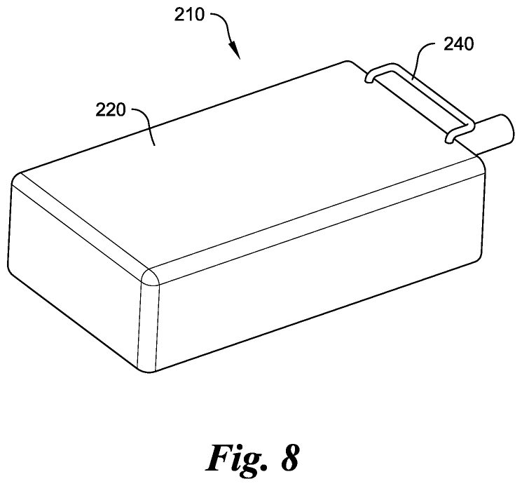

FIGS. 7 and 8 show another embodiment of the system, generally designated 210, of the presently disclosed technology. Similar or identical structure as between the embodiment of FIGS. 1-3 and the embodiment of FIGS. 7 and 8 is distinguished in FIGS. 7 and 8 by a reference number with a magnitude two hundred (200) greater than that of FIGS. 1-3. Description of certain similarities between the embodiments of FIGS. 1-3 and 4-6 and the embodiment of FIGS. 7 and 8 may be omitted herein for convenience and brevity only.

A distinguishing feature of the system 210 of the present embodiment is that the latching mechanism 240 comprises a pin structure to simply and firmly attach the plate 230 to the cover 220. The pins are removable, and can be configured to slide into position to retain the plate 230 and apply or maintain pressure to the seal.

The following exemplary embodiments further describe optional aspects of the presently disclosed technology and are part of this Detailed Description. These exemplary embodiments are set forth in a format substantially akin to claims (each set including a numerical designation followed by a letter (e.g., “A,” “B,” etc.), although they are not technically claims of the present application. The following exemplary embodiments refer to each other in dependent relationships as “embodiments” instead of “claims.”

1A. A method of cleaning valuable, sensitive, or electro-mechanical equipment, the method comprising:
inserting a plate permanently attached to a cable of the equipment into an opening of a cover;
placing a collar of a locking mechanism in a periphery groove of the plate and creating a water-tight connection between the plate and the cover; and
washing the combined cover, plate, and collar.

2A. The method of embodiment 1A, wherein washing occurs at ambient pressure inside and outside of the cover.

3A. The method of embodiment 1A or 2A, wherein the cover is formed of a flexible material.

1B. A system for washing valuable, sensitive, or electro-mechanical equipment includes a plate permanently attached to a cable of the equipment, and a cover configured to sealingly receive the plate within a single opening.

2B. The system of embodiment 1B, wherein the cover is formed of metal.

While the presently disclosed technology has been described in detail and with reference to specific examples thereof, it will be apparent to one skilled in the art that various changes and modifications can be made therein without departing from the spirit and scope thereof. It is understood, therefore, that the presently disclosed technology is not limited to the particular embodiments disclosed, but it is intended to cover modifications within the spirit and scope of the presently disclosed technology as defined by the appended claims.

I/We claim:

1. A system configured to protect valuable, sensitive, or electro-mechanical equipment during cleaning of the equipment, the equipment including a cable, the system comprising:
a cover having a single opening leading to an interior cavity, the covering being waterproof;
a plate permanently attached to the cable of the equipment, an outer periphery of the plate being approximately the same size as an inner periphery of the opening, the plate being configured to sealingly fit within the single opening; and
a latching mechanism configured to sealingly attach the plate to the cover.

2. The system of claim 1, wherein the equipment is a medical device.

3. The system of claim 2, wherein the cover is formed of a flexible, deformable, or compliant material.

4. The system of claim 3, wherein the plate includes a groove extending around a periphery thereof, and wherein at least a portion of the latching mechanism fits within the groove.

5. The system of claim 3, wherein the equipment is cleaned without pressurizing the interior cavity of the cover.

6. The system of claim 2, wherein the cover is rigid and formed of a metallic material.

7. The system of claim 2, wherein the plate includes an opening configured to sealing receive at least a portion of the cable therein.

8. The system of claim 7, wherein the latching mechanism includes two spaced-apart latches, each latch being configured to extend from the cover and engage a portion of the plate.

9. The system of claim 2, wherein the plate is permanently attached to the cable in a watertight manner by a seal or a gland.

10. The system of claim 1, wherein the cover is formed of at least one of silicone, a fluoroelastomer, nitrile, neoprene, ethylene propylene diene monomer (EPDM) rubber, or a thermoplastic elastomer (TPE).

11. The system of claim 1, wherein a valve is located in one of the plate or the cover.

12. A combination comprising:
a medical device including a cable; and
a system configured to protect at least a portion of the medical device during cleaning, the system comprising:
a cover having a single opening leading to an interior cavity, the cover being waterproof; and
a plate permanently attached to the cable of the medical device, an outer periphery of the plate being approximately the same size as an inner periphery of the opening; and
a latching mechanism configured to sealingly attach the plate to the cover,
wherein at least one of a connector or a port of the medical device is configured to be positioned in the interior cavity of the cover,
wherein the plate is configured to be inserted into and sealingly engage the single opening of the cover and enclose at least one of the connector or the port of the medical device within the interior cavity,
wherein the cable extends through the plate in a watertight manner, and
wherein the at least one of the connector or the port is cleaned without pressurizing the interior cavity of the cover.

13. The combination of claim 12, wherein the cover is formed of a flexible, deformable, or compliant material.

14. The combination of claim 12, wherein the cover is rigid and formed of a metallic material, and wherein the plate is configured to attach to the cover in a fluid-proof and gas-proof manner.

15. The combination of claim 14, wherein the plate is formed of a polymeric material.

16. The combination of claim 12, wherein the plate includes a pressurization valve or luer valve.

17. The combination of claim 12, wherein a valve is located in one of the plate or the cover, the valve being configured to test the integrity of the system.

18. The combination of claim 12, wherein the plate includes an opening configured to sealing receive at least a portion of the cable therein.

19. The combination of claim 18, wherein at least one latch is configured to extend from the cover and engage a portion of the plate.
